(19) United States
(12) Patent Application Publication
(10) Pub. No.: US 2005/0060775 A1
(43) Pub. Date: Mar. 17, 2005

(54) Production of human coagulation factor VIII from plant cells and whole plants

(76) Inventors: Brian S. Hooker, Kennewick, WA (US); Daniel B. Anderson, Pasco, WA (US); Johnway Gao, Richland, WA (US); Ziyu Dai, Richland, WA (US)

Correspondence Address: WELLS ST. JOHN P.S., 601 W. FIRST AVENUE, SUITE 1300, SPOKANE, WA, 99201, US

(21) Appl. No.: 10/863,969

(22) Filed: Jun. 8, 2004

Related U.S. Application Data
(63) Continuation-in-part of application No. 09/588,314, filed on Jun. 6, 2000.
Application No. 09/588,314 is a continuation of application No. 09/080,010, filed on May 14, 1998, now abandoned.

(51) Int. Cl. A01H 1/00; C12N 15/82

(52) U.S. Cl. 800/288; 435/468; 530/383

(57) ABSTRACT

The invention includes methods for production of a polypeptide having factor VIII activity by introduction of a polynucleotide construct into a plant cell. The construct includes an encoding sequence for a polypeptide of coagulation factor VIII or a functional variant thereof. The plant cell is cultured or regenerated into a plant and the polypeptide or functional variant of factor VIII is expressed therein. The invention also includes vectors, plant cells, plant tissues, plants and seeds containing a polynucleotide sequence encoding a functional variant of human coagulation factor VIII. The invention further includes a recombinant DNA molecule having a promoter which is functional in plants operably linked to a coding sequence which codes for a polynucleotide having coagulation factor VIII activity.

### RELATED PATENT DATA

This patent resulted from a continuation-in-part of U.S. patent application Ser. No. 09/588,314, filed Jun. 6, 2000, which is a continuation of U.S. application Ser. No. 09/080,010 which was filed May 14, 1998 and is now abandoned, each of which is incorporated herein by reference.

### GOVERNMENT RIGHTS

This invention was made with Government support under Contract DE-AC06 76RLO 1830 awarded by the United States Department of Energy. The Government has certain rights in the invention.

### TECHNICAL FIELD

The invention pertains to methods for production of a polypeptide having coagulation factor VIII activity. The invention additionally pertains to transgenic plants, transgenic plant cells, transgenic plant tissues, transgenic seeds and recombinant DNA molecules.

### BACKGROUND OF THE INVENTION

Factor VIII is a glycoprotein which occurs in plasma and has a critical role in blood coagulation. As initially translated, native factor VIII is a 2351 amino acid single chain protein. The protein consists of a 19 amino acid signal peptide and 6 domains commonly referred to as A1, A2, B (or beta), A3, C1 and C2. A mature form of the protein has a molecular weight of about 280 kDa and comprises a light chain and a heavy chain. The light chain has a molecular weight of approximately 80 kDa and comprises domains A3, C1 and C2. The heavy chain comprises domains A1, A2 and B and has a molecular weight of from about 90 to about 200 kDa.

When circulating in the blood, factor VIII is typically associated with a carrier protein known as von Willebrand factor. Activation of factor VIII occurs when thrombin and/or factor Xa proteolyzes the factor VIII protein which thereby induces dissociation from Von Willebrand factor. Once activated, factor VIIIa can in turn act in concert with additional factors to activate coagulation factor X, producing the activated factor Xa.

Hemophilia A is a condition which occurs due to a deficiency of functional factor VIII protein in plasma. Treatment of hemophilia A can typically comprise introduction of factor VIII in the form of isolated recombinant factor VIII protein from mammalian cell culture systems, or in the form of factor VIII concentrates derived from fractionated plasma. Production of factor VIII from plasma or mammalian cell culture systems can be difficult and cost prohibitive. Further, plasma derived factor VIII can contain contaminants and other unwanted impurities such as, for example, hepatitis A, B and C pathogens as well as parvovirus and human immunodeficiency virus (HIV) pathogens.

Many of the difficulties associated with human plasma derived factor VIII have been overcome by production of recombinant factor VIII in a variety of mammalian cell culture systems. Recombinant factor VIII has been produced in, for example, baby hamster kidney cell culture lines, Chinese hamster ovary (CHO) cell lines and monkey COS-7 cell lines. However, production of factor VIII using mammalian cell lines does not eliminate potential for transmission of pathogens to humans. Consequently, production from cell lines requires additional quality assurance testing and bio-safety trials to safeguard against pathogen transmission.

Due to the large size of the coding portion of the factor VIII gene (7.3 kb), production of factor VIII utilizing prokaryotic or lower eukaryotic hosts may be precluded. Use of prokaryotic host organisms to produce an active factor VIII may additionally be precluded due to the post translational modifications present in the mature factor VIII protein. Further, production of factor VIII in recombinant hosts other than mammalian cells has yet to be successfully completed.

It is desirable to develop alternative methods and systems for production of factor VIII which can be utilized for treatment of disease conditions and/or research purposes.

### SUMMARY OF THE INVENTION

In one aspect the invention encompasses methods for production of a polypeptide having factor VIII activity. A polynucleotide construct is introduced into a plant cell. The construct includes an encoding sequence for a polypeptide of coagulation factor VIII or a functional variant thereof. The plant cell is cultured and the polypeptide or functional variant of factor VIII is expressed in the cultured plant cell.

In one aspect the invention encompasses a vector which contains a polynucleotide sequence encoding a functional variant of human coagulation factor VIII.

In one aspect the invention encompasses a plant cell comprising a polynucleotide sequence encoding a functional variant of human coagulation factor VIII.

In one aspect the invention encompasses a plant seed comprising a polynucleotide encoding a functional variant of human coagulation factor VIII.

In one aspect the invention encompasses a plant tuber comprising a polynucleotide encoding a functional variant of human coagulation factor VIII.

In one aspect the invention encompasses a recombinant DNA molecule. The DNA molecule includes a promoter which is functional in plants and a coding sequence which codes for a polynucleotide having coagulation factor VIII activity. The coding sequence is operably linked to the promoter. The polypeptide is at least 70% identical to human coagulation factor VIII.

### BRIEF DESCRIPTION OF THE DRAWINGS

Preferred embodiments of the invention are described below with reference to the following accompanying drawings.

FIG. 1 is a schematic depiction of the construction and plasmid map of pSP64-FVIIIc.

FIG. 2 is a schematic depiction of the construction and plasmid map of pZD201.

FIG. 3 shows the result of a dot blot immuno-assay for T0 factor VIII plant transformants. S1 and S2 are positive control plasma-derived human factor VIII standards (American Diagnostica, Greenwich, Conn.). Leaf protein extract from untransformed Nicotiana tabacum cultivar SR1 was used as a negative control and is indicated by the designation SR.

FIG. 4 panels A and B show independent results of Western blot analysis of protein extracts from several T0 tobacco plant transformants with protein bands detectable in the plant extracts at positions corresponding to those that occur in samples of the native human protein. Lane SR1 is an untransformed control leaf extract sample; lanes FVIII are plasma-derived factor VIII standards. The remaining lanes in each panel correspond to samples extracted from independent T0 tobacco plants.

FIG. 5 shows the results of Western blot analysis of protein extracts from several T1 tobacco plants extracts with protein bands detectable in the plant extracts at positions corresponding to those that occur in samples of the native human protein. Lane F8 is a plasma-derived factor VIII standard. Lane SR1 is an untransformed control sample. Lane F13 is a transgenic plant control expressing human factor XIII A-subunit. The remaining lanes are samples of plant-derived factor VIII obtained from T1 plants. Bands observable at 240 kDa, 160 kDa and 140 kDa occur in both the plant derived and plasma-derived samples and correspond to products of proteolytic processing in the corresponding system.

FIG. 6 shows results of Western blot analysis of T0 tobacco plant extracts analyzing lower molecular weight fragments which correlate with positions of proteolytic fragments observed in the plasma-derived samples.

FIG. 7 shows results of Western blot analysis of protein extracts from several potato plant transformants as compared to untransformed control plants (FL1607) and plasma derived factor VIII standard (F8c; American Diagnostica, Greenwich, Conn.).

FIG. 8 shows a plasmid map of pBI221-rpl.

FIG. 9 shows a plasmid map of pBI221-rpl-factor VIII delta-B.

FIG. 10 shows results of Western blot analysis of extracts of tobacco protoplasts transformed to express B-domain deleted human coagulation factor VIII.

FIG. 11 shows results of an additional Western blot analysis of extracts of tobacco protoplasts transformed to express B-domain deleted human coagulation factor VIII.

FIG. 12 shows an independent Western blot analysis of extracts of tobacco protoplasts transformed to express B-domain deleted human coagulation factor VIII.

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

The invention encompasses utilization of plant cells, plant tissues, whole plants and plant seeds to produce coagulation factor VIII and functional variants thereof. For purposes of the description, the term “functional variant” can refer to a variant of a native factor VIII protein which exhibits measurable activity with respect to activation of coagulation factor X. A functional variant can be a functional fragment of factor VIII, a hybrid factor VIII protein, a factor VIII analog, or can comprise a sequence modification of one or more amino acid substitutions, insertions or deletions. A hybrid polypeptide as used herein can refer to a peptide produced from expression of a gene having an encoding sequence comprising two or more fused (in frame) nucleotide sequences. The two or more nucleotide sequences can be obtained from the same or differing organisms. Further, a variant can be a modification from a native factor VIII where the modification comprises, for example, alternative glycosylation, hydroxylation or other foreign moieties.

A number of functional factor VIII variants have been reported such as, for example, variants which lack a portion or all of the B-domain and factor VIII variants which have a portion of the human factor VIII sequence replaced with the corresponding porcine sequence. The present invention encompasses production of any of these functional variants in plants.

Although the invention encompasses production of factor VIII protein which comprises at least some non-human factor VIII sequences, in particular aspects it can be preferable that the factor VIII or factor VIII variant be highly homologous to human coagulation factor VIII. The sequence of human factor VIII is known in the art as disclosed in, for example, Wood et al. Nature 312:330 (1984) and U.S. Pat. No. 4,757,006 each of which is incorporated herein by reference. Human factor VIII genomic DNA or cDNA can be obtained or produced utilizing methods described in these incorporated references or by alternative methods.

The methodology of the invention can be utilized for production of recombinant human factor VIII having a sequence identical to the native human factor VIII, or a fragment thereof. Alternatively, a variety of modified biologically active factor VIII proteins can be produced utilizing well known techniques of in vitro mutagenesis to alter cloned DNA or cDNA.

In particular instances it can be preferable that the factor VIII molecules produced by the invention are at least 70% homologous to the native human factor VIII sequence. The terms sequence homology and sequence identity as used in the description refer to homology or identity between the sequence at issue as compared to the corresponding reference sequence (nucleic acid sequence or amino acid sequence). For purposes of the description, the term “homologous” with respect to an amino acid sequence can refer to an identity between sequences, or to a sequence having one or more conservative amino acid substitutions which do not measurably affect properties of the protein function.

In particular instances it can be preferable that the factor VIII variants of the invention are at least 70% identical to the human factor VIII sequence. In particular instances, the variants will have at least 80% and more preferably at least 90% amino acid sequence identity to the corresponding amino acid sequence of human factor VIII molecules. In particular instances, the factor VIII or factor VIII variant of the invention can have 100% identity to the corresponding human factor VIII sequence.

For purposes of the description the term “gene” refers to a DNA which includes an encoding region and one or more regions involved in regulation of expression of the coding sequence. The term gene can refer to a native gene (where native refers to naturally occurring nucleic acid), or can refer to a DNA molecule having at least some synthetic or recombinant portion. The term recombinant as used in the present description can refer to a nucleic acid molecule which is made at least in part by artificial combination of two or more segments.

As used herein the terms “transgenic”, “transfected”, or “transformed” can refer to any cell, tissue, organism or seed into which foreign or recombinant DNA has been introduced. When referring to plant cells, plant tissue, whole plants or other plant parts, the designation T0 can refer to the primary transformant, and the designation T1 can refer to the first generation produced from the primary transformant T0.

As used herein the term “expression” refers to transcription of a gene to produce corresponding RNA and translation of the mRNA to produce the gene product (i.e. peptide, polypeptide or protein), or a portion of the transcription and/or translation process.

The term “fragment” as used to describe nucleic acid, is utilized to refer to a portion of a nucleic acid sequence that is less than a full length. When utilized with reference to a protein, polypeptide or peptide, the term fragment indicates a portion of an amino acid sequence that is less than full length.

Methods of the invention can be utilized for production of full length coagulation factor VIII and/or biologically active variants of factor VIII. The variants encompassed by the invention include, but are not limited to, fragments and analogs as discussed above, including whole length proteins and fragments having one or more amino acid substitutions, insertions or deletions.

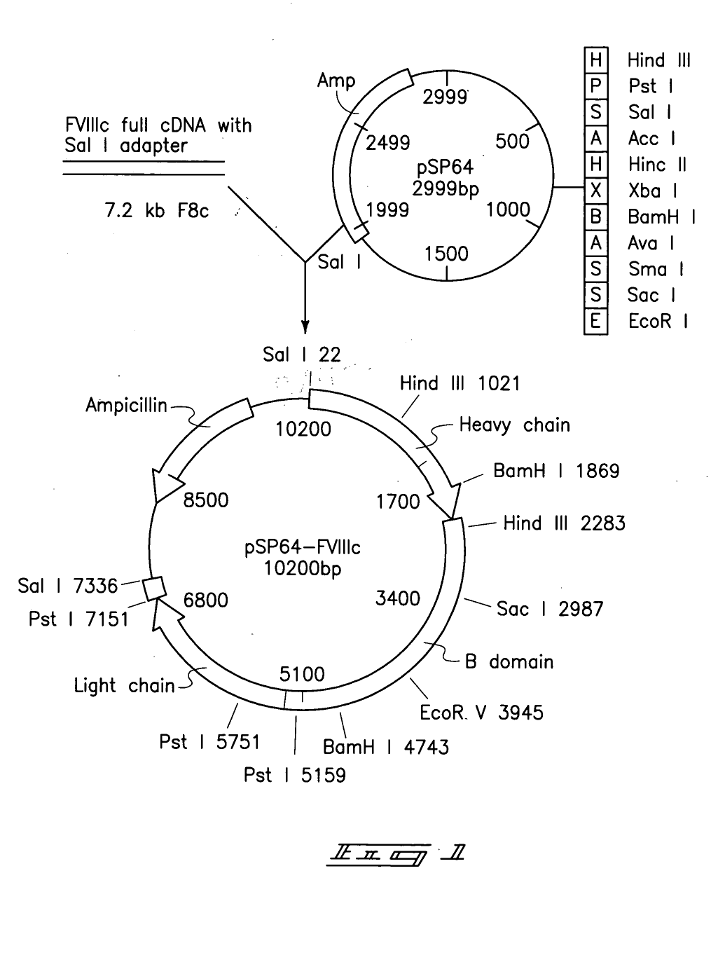

DNA encoding human factor VIII can be obtained by, for example, isolating the sequence from a genomic source such as, for example, human liver. Alternatively, an appropriate plasmid such as Escherichia coli plasmid pSP64-FVIII (ATCC No. 39812) can be obtained from the American Type Culture Collection (ATCC), Rockville, Md. A plasmid map of pSP64-FVIII is depicted in FIG. 1. The full length cDNA comprised by pSP64—FVIII encodes the entire 2332 amino acid protein sequence which includes the heavy chain A1 and A2 domains, the B-domain (beta-domain) and the light chain (A3, C1 and C2 domains). The cDNA additionally comprises the sequence encoding the native signal peptide (19 amino acids which are present at the N-terminus of the native human factor VIII protein as initially translated). Native human factor VIII protein as initially translated which contains the 19 amino acid signal peptide can be referred to as pre-coagulation factor VIII. Accordingly, the 7.3 kb cDNA contained in plasmid pSP64-FVIII can be referred to as pre-coagulation factor VIII cDNA.

The full length pre-coagulation factor VIII cDNA can be utilized in full length form or can be modified by, for example, site directed mutagenesis excision of one or more portions of the full length sequence, and/or hybridization by ligation of a corresponding sequence from an alternative organism. Although the invention is described as utilizing human factor VIII sequences, it is to be understood that the invention contemplates adaptation for utilization of factor VIII sequences from alternative organisms.

Upon obtaining the desired DNA encoding either the human factor VIII protein or a functional variant thereof, such can be utilized to create a recombinant DNA molecule for introducing into a plant. The recombinant DNA molecule can preferably comprise a promoter which is functional in plants, with the promoter being operably linked to the coding sequence. For purposes of the description, the term “promoter” refers to a minimal nucleic acid sequence located upstream or 5′ relative to the encoding nucleic acid sequence start codon sufficient to direct transcription of a nucleic acid sequence.

Promoters utilized for purposes of the present invention are preferably plant promoters where the term “plant promoter” refers to a promoter which is native to a plant or which is functional in plant cells. Promoters which can be utilized in accordance with the invention are not limited to a particular type and can be, for example, a constitutive promoter, an inducible promoter and/or a tissue specific promoter. Particular promoters which can be useful for purposes of the present invention include, but are not limited to, the cauliflower mosaic virus 35S (CaMV 35S) promoter, the tomato ribulose 5-bisphosphate carboxylase (RuBisCO) promoter, the tobacco ribosomal protein L34 promoter, the potato proteinase inhibitor I promoter, the potato aminotransferase promoter, the Schwanniomyces castellii alpha-amylase promoter and the Schwanniomyces castellii glucoamylase promoter.

The recombinant DNA molecule can additionally comprise a transcription terminator. Exemplary terminators which can be utilized for purposes of the present invention include, but are not limited to, the T7 transcription terminator, the T5 transcription terminator, and the nopaline synthase transcription terminator. Additional regulatory elements which can be incorporated into the recombinant gene include transcription enhancers, where a transcription enhancer refers to a nucleic acid element that can stimulate transcription of the recombinant gene. Exemplary enhancers include the octapine synthase enhancer, and the B-domain of the cauliflower mosaic virus 35S promoter.

Although the invention is described as providing any modification of the cDNA prior to operably linking to a desired promoter, it is to be understood that modification can be performed after linking of the encoding sequence to the promoter.

In particular aspects, the recombinant gene can be combined with one or more additional DNA sequences to form a larger DNA construct. In particular instances the genetic construct can comprise the recombinant gene inserted within vector DNA. An appropriate vector can be utilized for amplification, transfer and/or expression of the inserted recombinant factor VIII gene. Appropriate vectors for amplification of DNA, for DNA transfer and/or expression in plants are known by those skilled in the art. Such vectors can comprise, for example, additional genes or DNA sequences that can assist in amplification, selection, screening, and/or integration.

For purposes of the description, the term “amplification” of nucleic acid or nucleic acid sequences refers to the production of additional copies of the nucleic acid or sequence. Amplification can typically comprise polymerase chain reaction (PCR) technology.

The described genetic constructs can be introduced into plant cells, plant tissues or whole plants utilizing a variety of transformation techniques. Exemplary techniques which can be utilized for introduction of recombinant DNA in accordance with the invention include, but are not limited to, electroporation, pollen transformation, bacterial infection, binary bacterial artificial chromosome constructs, agitation with silicon carbide fibers, particle bombardment and chemical mediated uptake. The method of transformation utilized can depend upon the particular plant host. Plants which can be utilized as hosts for purposes of the invention are not limited to particular species. An appropriate plant host can be monocotyledonous or dicotyledonous. Exemplary plant hosts include but are not limited to potato, corn, tobacco, mustard, alfalfa, sunflower, wheat, collard, spinach, kale, canola, duckweed, carrot, rice, beet, cassava, soybean, poplar, cotton, onion and tomato.

Bacterial mediated transformation of a plant can comprise, for example, initial transformation of Agrobacterium tumefaciens utilizing, for example, the freeze-thaw method and subsequent introduction into a whole plant by, for example, leaf disks, or into a tissue or plant cell by co-cultivation. Where Agrobacterium is transformed utilizing a T1 plasmid, co-cultivation can result in a portion of the T1 plasmid (T-DNA) being transferred to and integrated into nuclear genomic DNA of the infected plant cells. Accordingly, a recombinant factor VIII gene can be integrated into plant genomic material and can subsequently be transcribed and translated in plant cells, tissues or whole plant. Agrobacterium mediated transformation of plants can be especially useful for introduction of recombinant factor VIII gene into plants such as tobacco, corn, tomato, sunflower, cotton, rapeseed, potato, poplar and soybean.

In particular aspects, micro-projectile bombardment can be utilized for transformation of plant cells. Particles such as gold or tungsten can be coated with DNA, such as recombinant factor VIII DNA which encodes functional factor VIII or variants thereof. The coated particles can then be accelerated toward plant cells to thereby transform the cells. This method can be utilized to stably transform cultures of plants such as maize, tobacco or onion for production of the factor VIII or factor VIII variant.

Pollen transformation can also be utilized to transform various plants such as, for example, tobacco and corn. Recombinant factor VIII DNA can be introduced into pollen grains by, for example, electroporation. The transformed pollen can then be utilized to produce a seed and eventually a plant. Any or all of the pollen, seed and plant can be screened for expression of recombinant factor VIII proteins. The pollen transfer method can be utilized to introduce the recombinant factor VIII gene into monocots as well as dicots.

Chemical mediated uptake of DNA by protoplasts can be utilized to introduce the recombinant DNA molecules of the invention into plant cells. Plant cell walls can be initially degraded enzymatically by methods known to those skilled in the art. The resulting protoplasts can be incubated in the presence of an appropriate vector for vector uptake, or can be incubated with a recombinant factor VIII gene in an absence of vector components for direct gene transfer. The incubation is conducted in the presence of polyethylene glycol which facilitates the transformation via direct insertion of the vector or gene. Where a vector is utilized, the vector can be a direct gene transfer vector or a Ti plasmid. The chemical mediated uptake methodology can be utilized for introducing the recombinant factor VIII gene into either monocot or dicot protoplasts.

Introduction of the factor VIII recombinant gene can be performed utilizing electroporation into plant protoplasts. In this method, the protoplasts are treated with an electrical pulse in the presence of the recombinant DNA to be introduced. Supercoiled or circular plasmid DNA, or linear DNA can be utilized for electroporation.

After successful transformation, the recombinant molecules of the invention can be expressed within plant cells or plant tissues in culture, or can be expressed in whole plants.

Where stable transformants are desired, plant regeneration can be achieved from any of a number of cells or tissues including tissue explants, tubers, seeds, callus culture, and protoplasts. Regeneration from callus tissue can be especially useful for monocots such as corn, rice, barley, wheat and rye, and dicots such as sunflower, soybean, cotton, rapeseed and tobacco. Regeneration of plants from protoplasts can be particularly useful where such protoplasts have been transformed via direct gene transfer methods including electroporation, PEG-mediated transformation, or micro-particle bombardment. Regeneration from protoplasts can be especially useful for plants such as: rice, tobacco, rapeseed and potato. Plants including tobacco, sunflower, tomato, rapeseed and cotton can be regenerated from tissues which have been transformed with A. tumefaciens mediated transformation.

Upon obtaining transgenic T0 plants, such can also be utilized to produce subsequent generation (T1) seed stock. The resulting seeds can be germinated to produce subsequent generations of plants.

Recombinant factor VIII gene expression from cell cultures, tissue cultures or whole plants in accordance with the invention can produce human coagulation factor VIII and functional variants. The resulting factor VIII and variants thereof exhibit biological factor VIII activity and antigenic interactions.

Post-translational proteolysis and other post-translational modifications of factor VIII have been well characterized and documented in the literature for a number of native systems. Many of these post-translational modifications including proteolytic processing have been shown to occur in cultured transgenic mammalian cells to produce biologically active factor VIII and functional variants of factor VIII. As shown below, proteolytic post-translational modification of recombinant factor VIII can also occur in plants to produce proteolytic fragments corresponding to the proteolytic fragments observed for factor VIII in native systems.

Utilizing methodology in accordance with the invention, factor VIII and functional variants similar or identical to those previously produced in native systems or cultured mammalian cells can be expressed from plant cells, plant tissue culture or whole plants. Although the invention is described and characterized with respect to specific types of variants of factor VIII, it is to be understood that the invention encompasses production of any of the functional variants reported in the literature which have been produced using alternative systems including mammalian cell systems and native systems. The invention also foresees production of additional factor VIII variants yet to be developed by adaptation of methodology of the present invention to express such variants in plants.

Factor VIII proteins can be collected from plant cells, plant tissue cultures, whole plants or parts of whole plants which express the recombinant factor VIII. Alternatively, under conditions where the heterologous factor VIII protein is excreted, the collection can comprise collection from culture media. As will be understood by those skilled in the art, collection and/or purification of factor VIII or a factor VIII variant from plant cells or plants can depend upon the particular expression system and particular variant being expressed. Separation and purification techniques can include, for example, protein precipitation, ultra filtration, affinity chromatography and or electrophoresis. In particular instances, molecular biological techniques known to those skilled in the art can be utilized to produce variants having one or more heterologous peptides which can assist in protein purification (purification tags). Such heterologous peptides can be retained in the final functional protein or can be removed during or subsequent to the collection/isolation/purification processing. Exemplary purification tags for purposes of the invention include but are not limited to the six-histidine tag, the V5 epitope tag and the myc epitope tag.

In addition to the aspects discussed above, the invention also encompasses co-expression of factor VIII with one or more additional recombinant proteins. For example, factor VIII can be co-expressed with von Willebrand factor. The co-expression of von Willebrand factor can be useful for stabilizing the factor VIII during expression and/or purification steps. Co-expression can utilize incorporation of multiple recombinant genes comprised by a single DNA construct or can be achieved by co-transforming with two or more recombinant DNA molecules.

In another aspect, the invention encompasses addition of one or more DNA sequences which encode, for example, a signal peptide or a peptide useful for purification purposes (discussed above) between the transcription promoter and the 5′ end of the coding sequence in a DNA construct. The encoded sequence can be used for purification purposes or can be a signal peptide to direct or localize the produced factor to a specific cellular organelle or can be a signal directing secretion of the protein. Exemplary signal peptides which may be utilized include tobacco PR-S signal peptide and the phytohemagglutinin signal peptide. In other aspects, one or more additional nucleic acid elements can be added between the promoter and regulatory elements or at the 3′ end of the encoding sequence to confer or enhance mRNA stability between transcription and translation events. An exemplary leader sequence which may be utilized to stabilize the transcript is the alfalfa mosaic virus RNA4 leader sequence.

### EXAMPLE 1

### Stable Transformation and Expression of Factor VIII in Plants

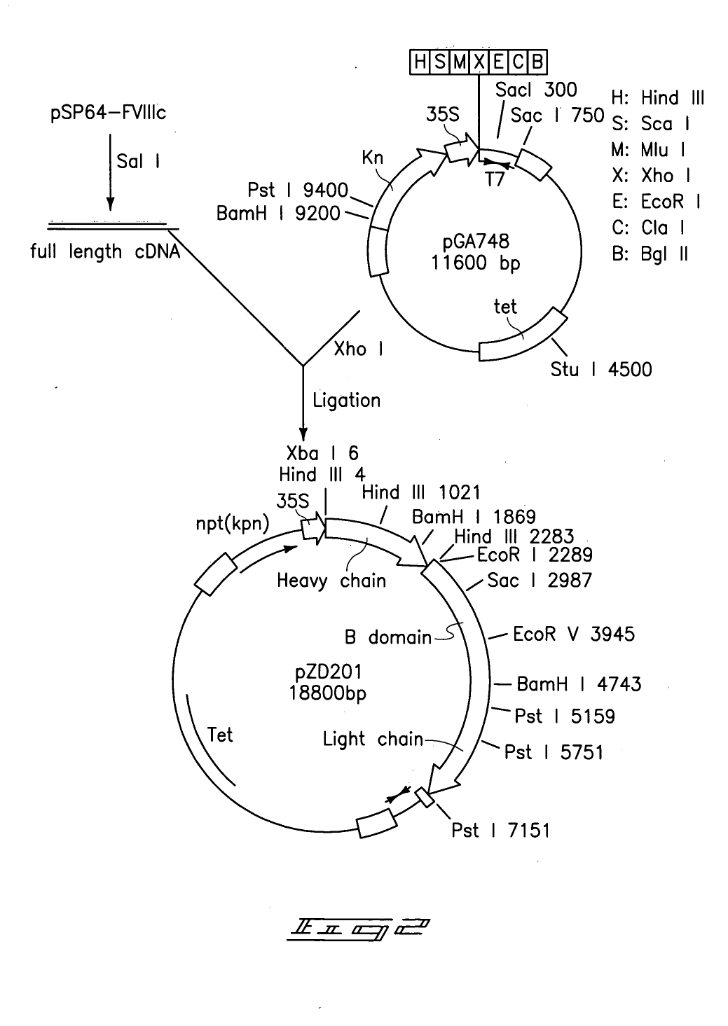

Escherichia coli plasmid pSP64-FVIII (ATCC No. 39812) shown in FIG. 1 was obtained from ATCC. The plasmid encodes the full length polypeptide of factor VIII cDNA derived from human fetal liver. The full length pre-coagulation factor VIII cDNA was excised utilizing Sal I restriction enzyme and was ligated into a compatible restriction enzyme site Xho I located between the CaMV 35S promoter and the T7 transcript terminator of the binary vector pGA748 t6 form the plasmid pZD201 as shown in FIG. 2. The pGA748 was directly transferred into Agrobacterium tumefaciens LBA4404 using the freeze-thaw method. The recombinant factor VIII gene was introduced into tobacco whole plants (by leaf disks) and into tobacco calli (by suspension culture) utilizing co-cultivation with the Agrobacterium. Over 200 samples of T0 transformants were taken from co-cultivated explants and suspension culture. Plants and calli were separately placed on kanamycin selective media. Upon obtaining positive transformants via kanamycin resistance screening, mature tobacco plants and calli were assayed for the presence of human coagulation factor VIII.

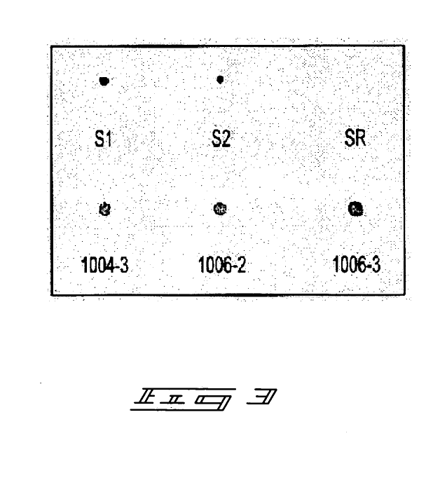

As shown in the dot blot assay of FIG. 3, coagulation factor VIII antigen was present in the leaf tissues of T0 whole plant transformants. As shown, each of the plants designated as 1004-3, 1006-2 and 1006-3 show strong factor VIII expression as observable by the immunoblotting technique. Control factor VIII standards are shown as S1 and S2 and negative control leaf protein from non-transformed N. tabacum is shown as SR.

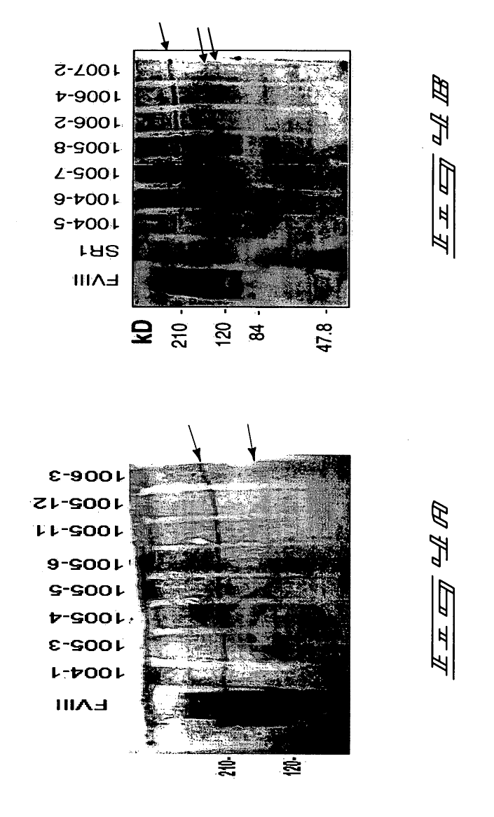

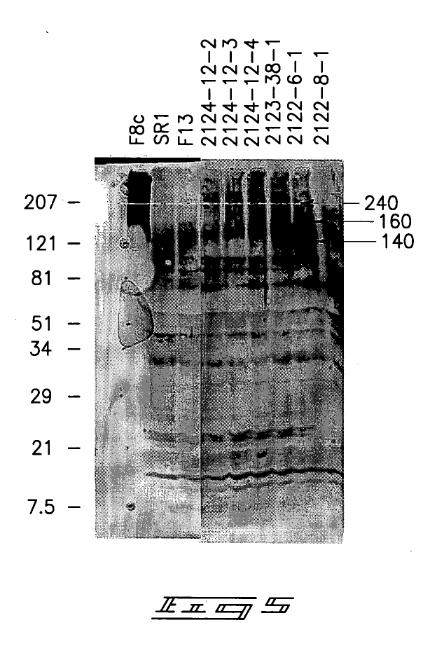

Upon completion of the preliminary dot blot immunoassay, T0 plants were self-pollinated resulting in T1 seedstock. The T1 seeds were subsequently germinated on a kanamycin selective media to obtain mature plants. Western immunoblot assays were performed on complete leaf protein extracts of the various plant lines, the results of which are presented in FIGS. 4 and 5.

As shown in FIG. 4, the predominant immunoreactive band for the transgenic T0 samples appears at approximately 240 kDa corresponding to the full length (non-proteolyzed) factor VIII protein. Additional bands which appear at 160 and 140 kDa correspond to the heavy chain of factor VIII and are consistent with results in the literature for bands which appear in native and mammalian cultures. The Western blot analysis presented in FIG. 5 shows immuno-reactive bands in the T1 samples comparable to those described in native and mammalian cell culture systems.

These results indicate that factor VIII expression in plants includes production of full length factor VIII as well as production of correctly post-translationally proteolyzed factor VIII subunits similar to those observed in native human system.

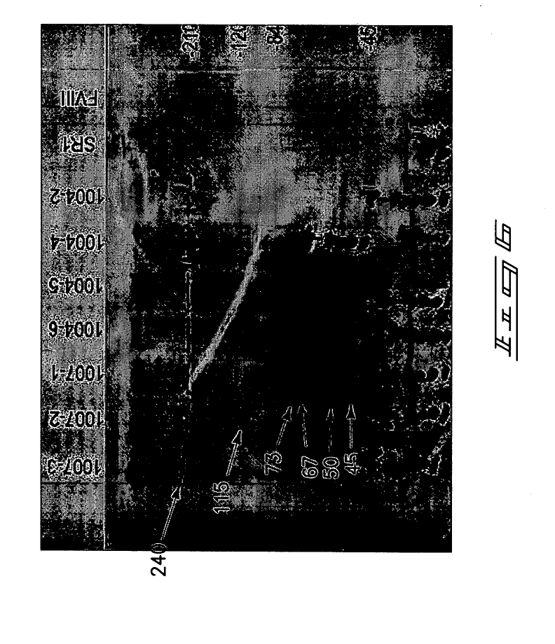

Lower molecular weight fractions from T0 plant extracts were also analyzed using Western blot techniques. The results of such Western blot assays are shown in FIG. 6. The factor VIII fragments observed utilizing Western blot analysis correspond to fragments of 73 kDa, 50 kDa and 43 kDa as well as 67 kDa and 45 kDa fragments which correspond to fragments of factor VIII produced by thrombin and factor Xa proteolytic cleavage of factor VIII as reported in the literature to occur in native and mammalian cell culture systems.

### EXAMPLE 2

### Factor VIII Activity Assay

Transgenic plant leaf material was harvested and total soluble protein was extracted utilizing standard techniques. Biological activity ability of recombinant human factor VIII was analyzed using the Coatest method. In the Coatest assay, a specific chromogenic substrate (MeO-CO-D-CHG-Gly-Arg-pNa) is utilized to determine activity. In this assay, the quantity of factor Xa generated from factor X due to factor VIII activity is measured.

The analysis of recombinant factor VIII comprised utilization of total protein samples from the transgenic plant material and an appropriate control comprised untransformed control plant total protein samples. The recombinant human factor VIII obtained from transgenic plant leaf material showed activity directly proportional to the amount of factor VIII present in the sample tested. Results of the Coatest assay are presented in Table 1.

TABLE 1
Coatest Assay of Plant Transformants

| | | Change in | Change in Absorbance |
|---|---|---|---|
| | | Absorbance | Compared to |
| | | Upon | Plant Control |
| | | addition of | ΔA₄₀₅[sample] − |
| Test | Plant Line | Factor Xₐ (A₄₀₅) | ΔA₄₀₅[control] |
| A | 1005-5 | 0.322 | 0.118 |
| A | 1005-6 | 0.269 | 0.065 |
| A | plant control | 0.204 | — |
| B | 1006-3 | 0.676 | 0.239 |
| B | plant control | 0.437 | — |
| C | plant control 1 w/ | 0.134 | 0.106 |
| | 5 ng FVIII | | |
| C | plant control 1 w/ | 0.176 | 0.148 |
| | 10 ng FVIII | | |
| C | plant control 1 w/ | 0.268 | 0.24 |
| | 30 ng FVIII | | |
| C | plant control 1 | 0.028 | — |
| C | plant control 2 w/ | 0.177 | 0.137 |
| | 5 ng FVIII | | |
| C | plant control 2 w/ | 0.222 | 0.182 |
| | 10 ng FVIII | | |
| C | plant control 2 w/ | 0.305 | 0.265 |
| | 30 ng FVIII | | |
| C | plant control 2 | 0.040 | — |

For each sample shown in the table, 1.5 mg of soluble plant protein was used. Tests A, B and C were performed on separate days and each required separate untransformed plant control. For tests A and B, the duration of incubation after addition of factor Xa was 5 minutes. For test C, the duration of incubation after factor Xa addition was 4 minutes. Additionally, test C included aliquots of factor VIII reference plasma standard added to two separate tobacco plant controls. The results from tests A and B show the presence of factor VIII pro-coagulant activity in tobacco plant lines 1005-5, 1005-6 and 1006-3 (as compared to increases in absorbance mediated by addition of factor VIII in test C). The level of activity observed in plant line 1006-3 corresponds to about 26 ng of factor VIII per 1500 μg of sample (based upon linear regression of calibration data from test C) or an expression level of 0.002% of extractable leaf protein. The results indicate that recombinant human factor VIII is correctly processed in plant resulting in pro-coagulant activity.

### EXAMPLE 3

### Potato Transformation for Expression of Coagulation Factor VIII

The plasmid pZD201 (as shown in FIG. 2) was directly transferred into Agrobacterium tumefaciens LBA4404 using the freeze-thaw method. The plasmid was then introduced into potato whole plants (by stem internodes) utilizing co-cultivation with the Agrobacterium to produce transformants. At least 50 specific samples of transformants were taken from the co-cultivation and were separately placed on kanamycin selected media. Upon obtaining positive transformants via kanamycin resistant screening, the mature potato plants were assayed for presence of human coagulation factor VIII.

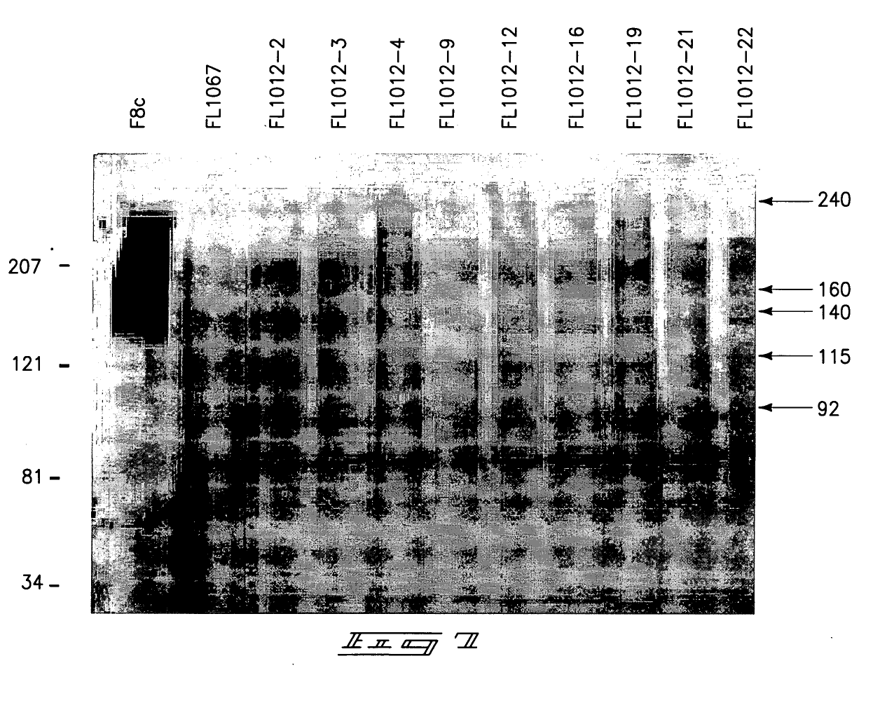

Protein immunoblotting was performed using extractable leaf protein and showed the presence of coagulation factor VIII antigen in leaf tissues of T0 whole plant transformants. Western blot analysis completed on leaf protein extracts of T0 plants are shown in FIG. 7. The results indicate the presence of immunoreactive bands corresponding to those previously identified for plasma-derived factor VIII. The band appearing at approximately 240 kDa corresponds to full length factor VIII heavy chain. Additional bands corresponding to factor VIII heavy chain appear at 90-200 kDa and corresponding to the light chain appear at approximately 80 kDa. These results indicate that plant-derived human factor VIII undergoes correct post-translational processing similar to that previously identified for plasma-derived factor VIII.

### EXAMPLE 4

### Production of a B-Domain Deletion Variant of Factor VIII in Plant protoplasts

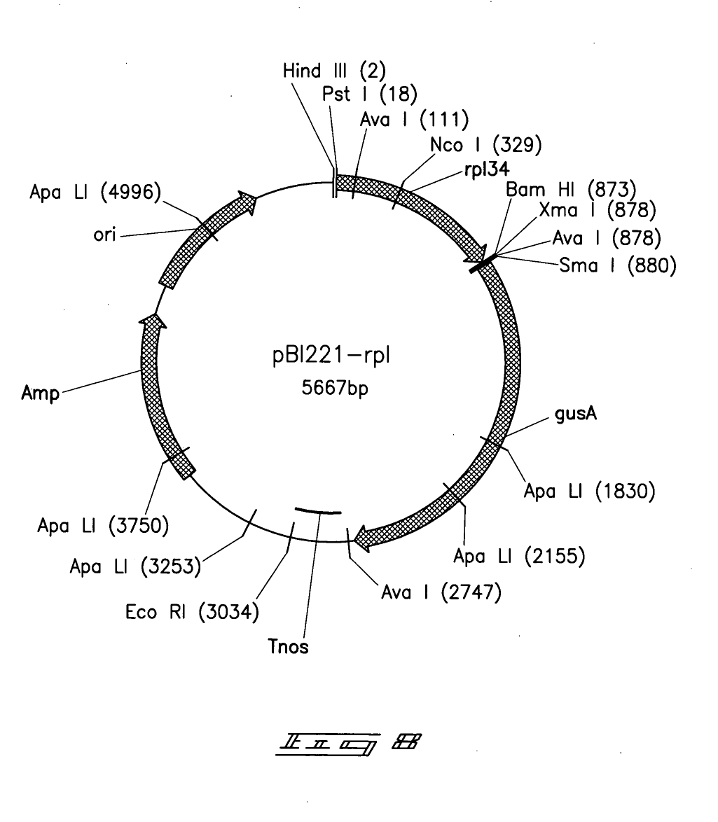

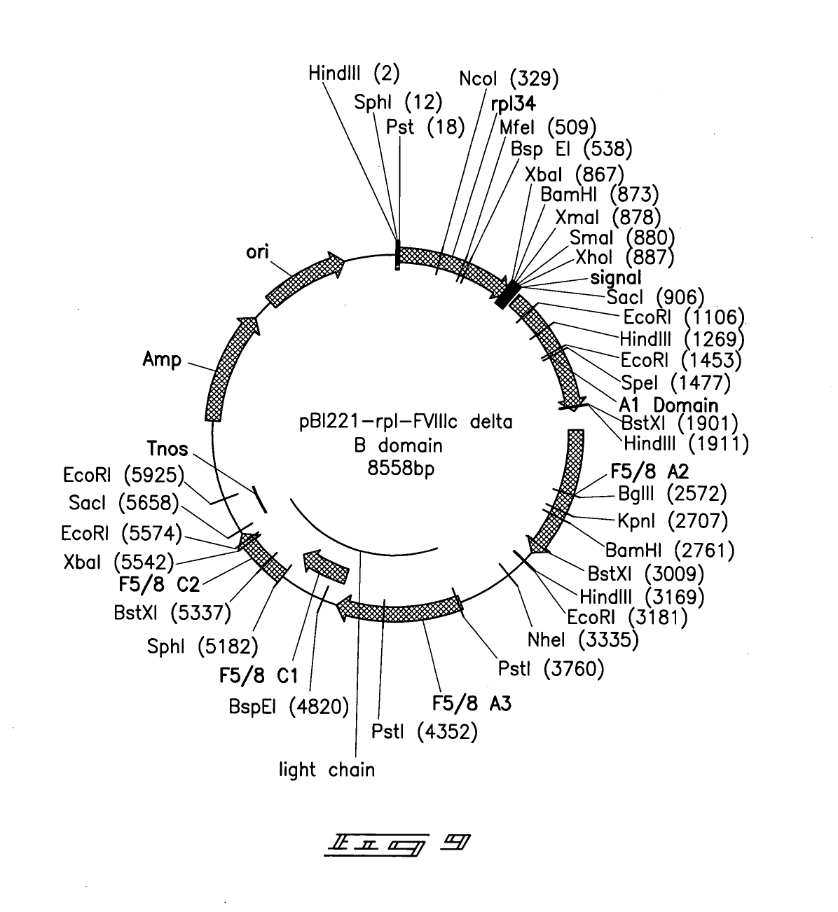

A transient expression vector for a B-domain deleted human factor VIII was constructed. The plasmid pBI221-rpl containing the rpL34 promoter (as shown in FIG. 8) was digested with Xma1 and Sac1 to remove the β-Glucuronidase (GUS) reporter gene. A restriction polylinker was created and the plasmid was digested with Nhe1 and Sac1. The C-terminal portion of the factor VIII coding region was amplified by PCR utilizing a forward primer having the sequence shown in SEQ ID NO: 1 and the reverse primer having the sequence shown in SEQ ID NO: 2. The resulting PCR product was subsequently ligated into the pBI221-rpl-Nhe1 vector (digested with Nhe1 and Sac1) and the presence of the Not1 site was used as negative selection. The N-terminal portion of human factor VIII was subsequently amplified using PCR with the forward primer having the sequence set forth in SEQ ID NO: 3 and the reverse primer having the sequence set forth in SEQ ID NO: 4. The N-terminal amplification resulted in a 2.54 kb product. The pBI221-rpl-FVIII vector was digested with Xho1 and Nhe1 and the N-terminal PCR product was inserted to create the pBI221-rpl-FVIII delta B-domain plasmid shown in FIG. 9.

A three day old NT1 tobacco cell suspension was utilized for preparation of protoplasts. The protoplasts were isolated by treating the suspension cells with a solution of pH 5.8 which contained 10 mg/l cellulase, 500 μg/ml pectolyase, and 0.4 M D-mannitol at 28° C. for 20 minutes at 100 rpm. The protoplasts where subsequently washed extensively with 0.4 M mannitol to remove cellulase and pectolyase. Approximately 1×10⁶ of the prepared protoplasts were resuspended in 0.5 ml of pH 5.5 electroporation buffer containing 2.38 mg/ml HEPES, 8.76 mg/ml NaCl, 735 μg/ml CaCl₂ and 0.4 M D-mannitol.

20 μg of supercoiled plasmid DNA and 10 μg salmon sperm DNA was added as carrier DNA to the protoplasts which were then electroporated utilizing a 300 volt pulse. The treated protoplasts were subsequently transferred into 7 ml of protoplast culture medium (modified Murashige and Skoog (MS)) containing 4.3 mg/ml MS salt supplemented with 3% sucrose, 0.18 mg/ml KH₂PO₄, 0.1 mg/ml inositol, 1 μg/ml thiamine hydrochloride, and 0.2 μg/ml 2.4-dichlorophenoxyacetic acid (2.4-D) and 0.4 M D-mannitol. The cultured protoplasts were collected utilizing centrifugation and were resuspended in 100 μl of extraction buffer (50 mM Tris-HCl pH8.3, 227 mM NaCl, 1 mg/ml bovine serum albumin, and 1 mg/ml sodium azide). Protein samples were extracted utilizing sonication of the protoplasts three times for 8 seconds at 30-second intervals. The protein samples were harvested by centrifugation of the sonicated mixture at 15,000 g for 5 minutes. The supernatant was saved and protein concentration was measured using the BIO-RAD® (Bio-Rad Laboratories, Inc. Hercules Calif.) reagent protein assay according to the Bradford method (Bradford, MM. A rapid and sensitive method for the quantitation of microgram quantities of protein utilizing the principle of protein-dye binding. Analytical Biochemistry 72: 248-254.1976).

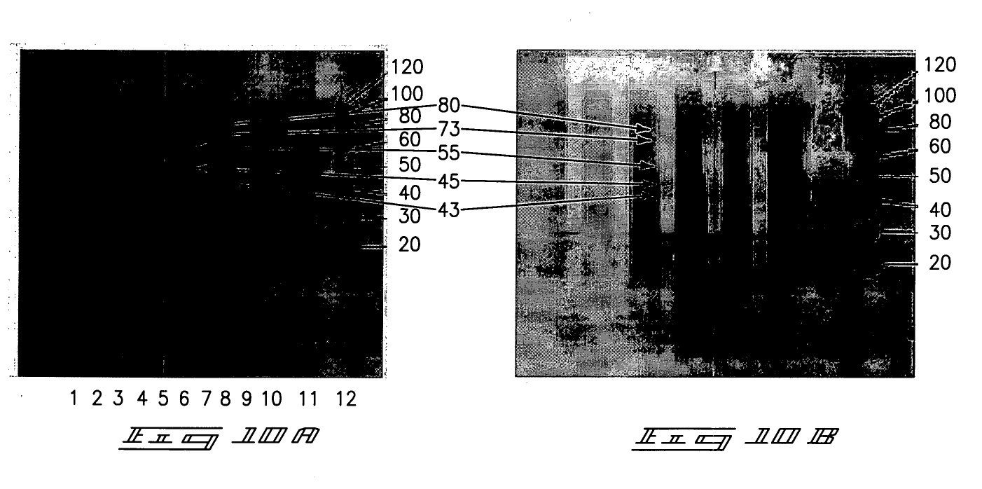

Western blot analysis was completed on protoplast extract and media samples as shown in FIGS. 10-12. The results shown in FIG. 10 are from four independent cultures, with lanes 3, 5, 7 and 9 corresponding to individual protoplast extracts and lanes 4, 6, 8 and 10 corresponding to individual media samples. Lane 1 is a control protoplast sample, lane 2 is a control media sample, lane 11 is a factor VIII standard and lane 12 is a molecular weight marker standard. Bands observable in test samples at 80 kDa correspond to the A3-C1-C2 fragment plus a small portion of the B-domain retained during cloning; bands at 73 kDa correspond to the A3-C1-C2 fragment; bands at 55 kDa correspond to the A2 fragment having a retained portion of the B-domain; bands observable at 45 kDa correspond to the A1 fragment truncated by factor Xa-like proteolysis; and bands at 43 kDa correspond to the A2 fragment.

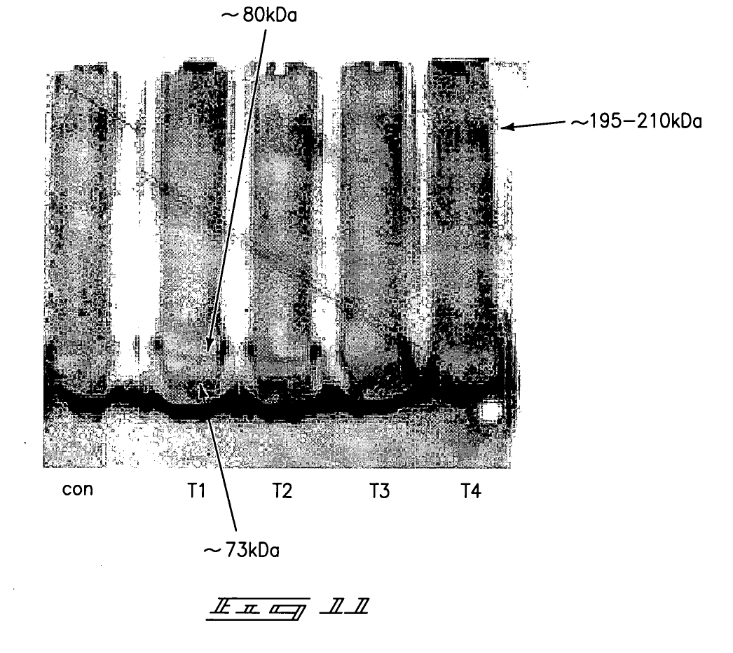

The lanes shown in FIG. 11 correspond to a protoplast control (lane 1); and individually electroporated protoplast lines T1-T4 (lanes 2-5). The results presented in FIG. 11 show bands observable at 195 kDa corresponding to an intact factor VIII protein lacking the entire B-domain; at 80 kDa corresponding to the A3-C1-C2 fragment plus a small portion of the B-domain retained during cloning; and at 73 kDa corresponding to the A3-C1-C2 fragment.

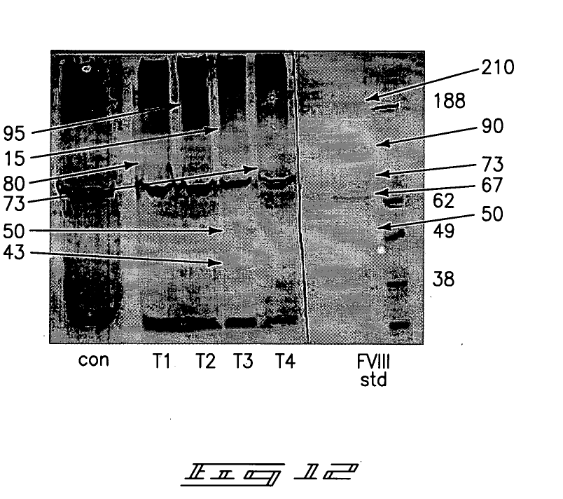

The lanes shown in FIG. 12 correspond to a protoplast control (lane 1); individually electroporated protoplast lines T1-T4 (lanes 2-5), and a set of molecular weight standards (lane 6). FIG. 12 also shows the reactive bands for transgenic protoplast extracts corresponding to an intact factor VIII lacking the entire B-domain (195 kDa), and factor VIII fragments (115 kDa, corresponding to the A1-A2 fragment plus a small portion of the B-domain; 80 kDa, corresponding to the A3-C1-C2 fragment plus a small portion of the B-domain; 73 kDa, corresponding to the A3-C1-C2 fragment; 50 kDa, corresponding to the A1 fragment; and 43 kDa, corresponding to the A2 fragment). FIG. 12 additionally compares the extracts to a plasma-derived coagulation factor VIII standard (FVIII std).

### EXAMPLE 5

### Production of a Beta-Deletion Variant of Factor VIII in Whole Plants and Calli

The B-domain deletion factor VIII coding region shown in FIG. 8 is excised and ligated into compatible restriction enzyme sites located between the CaMV 35S promoter and T7 transcript terminator of a binary vector pGA748. The plasmid is directly transferred into Agrobacterium tumefaciens LBA4404 using the freeze-thaw method. The plasmid is introduced into tobacco whole plants (by leaf disks) and calli (by suspension culture) utilizing co-cultivation with Agrobacterium to produce transformants. Upon obtaining positive transformants via kanamycin resistance screening of mature tobacco plants and calli, the presence and activity of B-domain deletion coagulation factor VIII are verified utilizing immuno-blotting and Coatest assay respectively.

### EXAMPLE 6

### Production of a Functional A2 Domain Substituted Factor VIII in Plants and Calli

The Escherichia coli plasmid pSP64—FVIII (ATCC No. 39812) containing the gene encoding full length polypeptide of factor VIII cDNA derived from human fetal liver is obtained as described above. The full length factor VIII sequence is excised and inserted into an appropriate cloning vector. The A2 domain in human factor VIII is removed and is replaced with an analogous porcine sequence. It can be advantageous to replace the A2 domain with porcine sequence to eliminate an inhibitory epitope as previously described in the literature. The cDNA encoding the porcine A2 domain is obtained following PCR of reverse-transcribed porcine spleen mRNA isolated using appropriately designed primers based on the porcine in human factor VIII sequences.

The human A2 domain is removed using site directed mutagenesis which excises nucleotides 1169-2304 of the human sequence (corresponding to the A2 domain). An appropriate restriction site for insertion of the porcine analogous sequence is introduced. The analogous porcine sequence is amplified utilizing a vector comprising the porcine A2 domain. The porcine A2 sequence is inserted directly into the corresponding restriction site into the A2 domainless human factor VIII. The A1/A2 and A2/A3 junctions are corrected to restore precise thrombin cleavage and flanking sequences utilizing site directed mutagenesis. The resulting construct has the human A2 domain exactly substituted with the analogous porcine A2 domain. Sequence identity is confirmed via read-through sequencing reactions.

The resulting hybrid pre-coagulation factor VIII cDNA is excised with Sal I restriction enzyme and sequentially ligated into the compatible restriction enzyme site Xho I located between the CaMV 35S promoter and the T7 transcription terminator of binary vector pGA748. The plasmid is directly transferred into Agrobacterium tumefaciens LBA4404 using the freeze-thaw method. The transferred plasmid is introduced into tobacco whole plants (by leaf disks) and calli (by suspension culture) by co-cultivation with Agrobacterium to produce transformants. Upon obtaining positive transformants via kanamycin resistance screening, mature tobacco plants and calli are assayed to verify the presence of hybrid coagulation factor VIII using protein immunoblotting and activity utilizing the Coatest assay.

### EXAMPLE 7

### Production of an Inactivation Resistant Coagulation Factor VIII in Plants

A functional factor VIII variant which has sequences of the native protein that allow inactivation by thrombin or protein C removed is produced in plants and calli. The coding region of the B-deletion factor VIII variant shown in FIG. 8 is utilized for modification and subsequent production of a factor VIII variant protein having amino acids 795-1685 of the native sequence deleted, having arginine-336 replaced with isoleucine, arginine-562 replaced with lysine, and arginine-740 replaced with alanine. The coding region encoding the B-deletion variant is excised and is subsequently ligated into the Xho I site between the CaMV 35S promoter and the T7 transcription terminator of the pBI221 cloning vector. Thrombin and protein C inactivation sites present in the native sequence are removed utilizing missense mutation technology to produce a single-chain protein which is activated by a single proteolytic cleavage after arginine-372. Site directed mutagenesis is utilized to replace arginine at amino acid positions 336, 562 and 740 with isoleucine, lysine and alanine respectively.

The resulting recombinant gene is excised and is ligated into the T-DNA region of the T1-plasmid pGA748 which is subsequently directly introduced into Agrobacterium tumefaciens LBA4404 utilizing the freeze-thaw method. Co-cultivation with the Agrobacterium tumefaciens is utilized to introduce the recombinant gene into tobacco whole plants (by leaf disks) and calli (by suspension culture) to produce transformants. Positive transformants are obtained utilizing kanamycin resistance screening. Presence of the recombinant inactivation-resistant factor VIII variant in the resulting mature tobacco plants and calli is verified using immunoblot techniques, and activity of the resulting factor VIII variant is detected utilizing the Coatest assay, as described above.

### EXAMPLE 8

### Production of a Functional C2 Domain Substituted Factor VIII in Plant

A functional factor VIII variant having the human C2 domain replaced with the corresponding porcine C2 domain sequence is produced in plants and calli. As described in the literature, replacement of the human C2 sequence with the porcine C2 domain can be advantageous due to elimination of an inhibitory epitope present in the human C2 domain. The full length factor VIII sequence is excised from pSP64-FVIII (ATCC No. 3981, described above), and is subsequently inserted into cloning vector pBI221 having a GUS insert and a Not I site at the 3′ end of the GUS gene. A porcine factor VIII cDNA, corresponding to a portion of the 3′ end of the C1 domain and including the entire C2 domain is obtained from porcine spleen total RNA utilizing primers based upon known porcine factor VIII sequence, and is cloned into pBluescript utilizing real-time PCR. The resulting pBluescript construct and the pBI221-human factor VIII (hFVIII) construct (described above) are utilized as templates during splicing-by overlap extension mutagenesis to construct a fusion product having the human C1 domain and the porcine C2 sequence. The fusion product is excised from the pBluescript construct utilizing Apa I and Not I and is subsequently ligated into Apa I/Not. I digested pBI221-hFVIII to result in the recombinant gene encoding the pre-coagulation factor VIII hybrid having the human C2 domain replaced with the corresponding porcine sequence.

The resulting hybrid pre-coagulation factor VIII cDNA is excised from the pBI221 construct with the Sal I restriction enzyme and is subsequently ligated into the Xho I site between the CaMV 35S promoter and the T7 transcription terminator of binary vector pGA748. The binary vector is then transferred directly into Agrobacterium tumefaciens utilizing the freeze-thaw method, and is subsequently introduced into tobacco whole plants (by leaf disks) and into calli (by suspension culture) utilizing co-cultivation techniques. Kanamycin resistance screening is utilized to obtain positive transformants. Transformed mature plants and calli are analyzed utilizing immunoblot techniques to verify the presence of the factor VIII porcine-C2 hybrid. The activity of the hybrid protein from plant is detected utilizing the Coatest assay.

In addition to the examples presented above, it is to be understood that the invention contemplates production of alternative functional factor VIII variants from plants. Examples of alternative variants include combinations of the deletions and/or substitutions set forth in the examples. Further, the invention contemplates adaptation of the described methodology to produce in plants any of the factor VIII variants described in the literature as being producible in mammalian cell culture systems.

It can be advantageous to produce coagulation factor VIII and/or functional variants of factor VIII from plants to reduce costs and safety risks relative to alternative production methods. Costs for production of factor VIII from transgenic plants in accordance with the invention can be from two to four orders of magnitude lower than corresponding cost of production from mammalian cell processes. In contrast to risks associated with some mammalian-derived protein formulations, plant-based production of factor VIII can be utilized to produce non-pathogenic and non-oncogenic factor VIII formulations.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

1. A method of producing a polypeptide having coagulation factor VIII activity, comprising:
providing a DNA construct comprising a promoter operably linked to a polynucleotide sequence encoding the polypeptide having coagulation factor VIII activity;
introducing the construct into a plant cell; and
expressing the polynucleotide sequence in the plant cell.

2. The method of claim 1 wherein the polynucleotide sequence encodes the entire human factor VIII protein and wherein the expressing produces at least some proteolized fragments of the human factor VIII protein, the fragments including at least one of intact light chain and intact heavy chain.

3. The method of claim 1 wherein the polynucleotide sequence encodes the A1, B, A3, C1 and C2 domains of human factor VIII protein and the A2 domain of porcine factor VIII.

4. The method of claim 1 wherein the polynucleotide sequence encodes a factor VIII variant which lacks a portion or an entirety of the B-domain.

5. The method of claim 1 wherein the polynucleotide sequence encodes an inactivation resistant factor VIII protein.

6. The method of claim 1 wherein the polynucleotide sequence encodes the A1 A2, B, A3, and C1 domains of human factor VIII protein and the C2 domain of porcine factor VIII.

7. The method of claim 1 wherein the polynucleotide sequence encodes the A1, B, A3, and C1 domains of human factor VIII protein and the A2 and C2 domains of porcine factor VIII.

8. The method of claim 1 further comprising regenerating the plant cell to produce a plant wherein the polynucleotide sequence is expressible in the plant.

9. A method for the production of a polypeptide comprising:
introducing into a plant cell a polynucleotide construct comprising an encoding sequence for a polypeptide, the polypeptide being a functional variant of coagulation factor VIII;
culturing the plant cell; and
expressing the polypeptide in the cultured plant cell.

10. The method of claim 9 wherein the polypeptide comprises the intact factor VIII light chain and at least a portion of the factor VIII heavy chain.

11. The method of claim 9 wherein the polypeptide lacks at least a portion of the factor VIII beta-domain.

12. The method of claim 9 wherein the polypeptide comprises human factor VIII light chain sequence.

13. The method of claim 9 wherein the polypeptide comprises a porcine coagulation factor VIII A2 domain.

14. The method of claim 9 wherein the polypeptide comprises a porcine coagulation factor VIII C2 domain.

15. The method of claim 9 wherein the polypeptide comprises an inactivation resistant coagulation factor VIII variant.

16. The method of claim 9 further comprising collecting the expressed polynucleotide, wherein the collecting comprises at least one of extraction, affinity chromatography, precipitation, ultrafiltration, and electrophoresis.

17. The method of claim 9 wherein the plant cell is from a plant selected from the group consisting of potato, tobacco, corn, mustard, alfalfa, sunflower, wheat, collard, kale, spinach, beet, cassaya, canola, duckweed and carrot.

18. The method of claim 9 wherein the plant cell is comprised by a plant tissue, and wherein the culturing comprises culturing the plant tissue.

19. The method of claim 9 wherein the plant cell is comprised by a plant tissue, and further comprising regenerating a plant from the plant tissue.

20. The method of claim 9 further comprising regenerating the plant cell to produce a whole plant.

21. The method of claim 9 wherein the culturing occurs in vitro.

22. The method of claim 9 wherein the introducing comprises at least one of electroporation, pollen transformation, bacterial infection, binary bacterial artificial chromosome constructs, agitation with silicon carbide fibers, particle bombardment, and chemical mediated uptake.

23. The method of claim 9 further comprising, constructing a vector comprising the encoding sequence prior to the introducing.

24. A Ti vector comprising a polynucleotide sequence encoding a functional variant of human coagulation factor VIII.

25. The Ti vector of claim 24 wherein the functional variant comprises at least 70% homology to human coagulation factor VIII.

26. The Ti vector of claim 24 wherein the functional variant comprises at least 70% identity to human coagulation factor VIII.

27. The Ti vector of claim 24 wherein the functional variant lacks at least a portion of the coagulation factor VIII beta-domain.

28. The Ti vector of claim 24 wherein the functional variant is a hybrid polypeptide comprising a portion of the porcine coagulation factor VIII sequence.

29. The Ti vector of claim 24 wherein the functional variant is an inactivation resistant factor VIII protein.

30. A plant cell comprising a polynucleotide sequence encoding a functional variant of human coagulation factor VIII.

31. The plant cell of claim 30 wherein the plant cell is in suspension culture.

32. The plant cell of claim 30 wherein the plant cell is comprised by a plant tissue.

33. The plant cell of claim 30 wherein the plant cell is comprised by a whole plant.

34. The plant cell of claim 30 wherein the polynucleotide sequence is incorporated into the genome.

35. A plant seed comprising a polynucleotide encoding a functional variant of human coagulation factor VIII.

36. The seed of claim 35 wherein the functional variant has at least 70% homology to human coagulation factor VIII.

37. A recombinant DNA molecule comprising:
a promoter functional in plants;
a coding sequence which codes for a polypeptide having coagulation factor VIII activity, wherein the polypeptide is at least 70% identical to human coagulation factor VIII, the coding sequence being operably linked to the promoter.

38. The recombinant DNA molecule of claim 37 wherein the recombinant DNA molecule is double stranded.

39. The recombinant DNA molecule of claim 37 wherein the polypeptide lacks at least a portion of the coagulation factor VIII beta-domain.

40. The recombinant DNA molecule of claim 37 wherein the polypeptide comprises A1, A3, C1 and C2 amino acid sequences from human coagulation factor VIII and A2 porcine coagulation factor VIII amino acid sequence.

41. The recombinant DNA molecule of claim 40 wherein the polypeptide further comprises the human coagulation factor VIII beta domain amino acid sequence.

42. A transgenic plant cell containing the recombinant DNA molecule of claim 37.

43. A transgenic plant comprising the plant cell of claim 42.

44. A transgenic plant seed comprising the recombinant DNA molecule of claim 37.

45. A transgenic plant tuber comprising the recombinant DNA molecule of claim 37.


SEQUENCE LISTING (4 sequences)

SEQ ID NO: 1 - DNA, 33 bp, Artificial sequence
  oligonucleotide primer
ctagctagcgatcctcttgcttgggataaccac

SEQ ID NO: 2 - DNA, 35 bp, Artificial sequence
  oligonucleotide primer
ttcgagctctcagtagaggtcctgtgcctcgcagc

SEQ ID NO: 3 - DNA, 33 bp, Artificial sequence
  oligonucleotide primer
ccgctcgagatgcaaatagagctctccacctgc

SEQ ID NO: 4 - DNA, 37 bp, Artificial sequence
  oligonucleotide primer
ctagctagctcgcaagagcatcaacaaatcactagag